(19) United States
(12) Patent Application Publication
(10) Pub. No.: US 2002/0098164 A1
(43) Pub. Date: Jul. 25, 2002

(54) Treatment of tumor metastases and cancer

(76) Inventors: H. Paul Redmond, Wilton (IE); Rolf W. Pfirrmann, Lucerne (CH)

Correspondence Address: ROTHWELL, FIGG, ERNST & MANBECK, P.C., 1425 K STREET, N.W., SUITE 800, WASHINGTON, DC, 20005, US

(21) Appl. No.: 09/983,279

(22) Filed: Oct. 23, 2001

Related U.S. Application Data
(60) Provisional application No. 60/243,409, filed on Oct. 27, 2000.

(51) Int. Cl. A61K 38/20; A61K 31/54

(52) U.S. Cl. 424/85.2; 514/222.5

(57) ABSTRACT

Tumor metastases in cancer patients are inhibited by administration of a combination therapy including effective amounts of Interleukin-2 and a methylol transfer agent such as taurolidine, taurultam or mixtures thereof.

### CROSS-REFERENCE TO RELATED APPLICATION

The present application claims the benefit of U.S. Provisional Application Ser. No. 60/243,409, filed Oct. 27, 2000.

### BACKGROUND OF THE INVENTION

1. Field of the Invention

The present invention relates to the field of treating tumor metastases and cancer.

2. Description of the Background Art

Interleukin-2 (IL-2) is an agent which has been suggested for inhibiting tumor cell growth. However, administration of IL-2 to patients presents severe toxicity problems, since IL-2 elicits an extremely strong systemic inflammatory response syndrome (SIRS) reaction in patients. Toxicity of IL-2 is so severe that approximately 70% of patients cannot tolerate treatment.

Additionally, a common problem in patients undergoing cancer treatment is tumor recurrence or metastasis.

Thus, despite the advances in cancer treatment, there remains a significant need in the art for new and improved cancer treatment therapies.

### SUMMARY OF THE INVENTION

In accordance with the present invention, tumor metastasis is inhibited in a cancer patient by administering to said patient a combination therapy comprising effective amounts of IL-2 and a methylol transfer agent.

### DETAILED DESCRIPTION OF THE INVENTION

It has surprisingly been found that methylol transfer agents such as taurolidine and taurultam reduce or substantially eliminate the severe toxicity and side effects of IL-2 in a combination therapy for inhibiting tumor metastases and treating cancer in patients, while it has unexpectedly been found that the efficacy of IL-2 is actually enhanced by the methylol transfer agents in the combination therapy of the present invention.

IL-2 when used in accordance with the present invention includes natural or recombinant Interleukin-2, or biologically active derivatives or substantial equivalents thereof.

Methylol transfer agents include methylol-containing compounds such as taurolidine and taurultam. The compounds taurolidine and taurultam are disclosed in U.S. Pat. No. 5,210,083. Other suitable methylol-containing compounds may be found among those identified in PCT Publication No. WO 01/39763. Particularly preferred methylol transfer agents for utilization in accordance with the present invention are taurolidine, taurultam, biologically active derivatives thereof and mixtures thereof.

Particularly preferred embodiments involve treatment of cancers selected from the group consisting of malignant melanoma and renal cancer, and inhibition of tumor metastases thereof. For example, the combination therapy of the present invention has been found to be particularly effective in inhibiting metastatic malignant melanoma and metastatic renal cell carcinoma.

Other cancers to which the combination therapy of the present invention is effective may include other carcinomas, sarcomas or lymphomas. Cancers to which the present invention may be applicable include glioma, neuroblastoma, astrocytoma, carcinomatous meningitis, breast cancer, ovarian cancer, colon cancer, prostate cancer, pancreatic cancer, central nervous system (CNS) cancer, liver cancer, lung cancer, gastric cancer, esophageal cancer, urinary bladder cancer, leukemia, lymphoma, melanoma, renal cell cancer and metastases thereof.

Effective daily dosage amounts of IL-2 may comprise pharmaceutical dosage units within the range of 1,000,000-100,000,000 units (U) IL-2 per m² body surface area. Dosage amounts of IL-2 also may be found within the range of 100,000-1,000,000 U per kilogram body weight. Dosage amounts of IL-2 further may be found within the range of 0.1-100 micrograms IL-2 per kilogram body weight.

Effective dosage amounts of a methylol transfer agent in accordance with the present invention may comprise pharmaceutical dosage units within the range of about 0.1-1,000 mg/kg. Preferred dosages may be in the range of about 10-20 grams taurolidine, taurultam or a mixture thereof, per administration.

Pharmaceutical dosage units of the combined therapy of the present invention may be administered by any suitable route, which include oral, topical or peritoneal administration, e.g., subcutaneously, intraperitoneally, intramuscularly, or intravenously, e.g., by infusion or injection.

In preferred embodiments, 250 ml of taurolidine 2% solution is administered by intravenous infusion about 1-6 times per day, more preferably about 2-4 times per day, during a treatment period, concurrently with administration of about 10,000,000-40,000,000 units m² IL-2 by intravenous infusion per day during the treatment period.

The present invention also is directed to a combination of IL-2 and a methylol transfer agent, in effective amounts for simultaneous, separate or sequential use for inhibiting tumor metastasis in a cancer patient. The invention also is directed to pharmaceutical combinations including pharmaceutical dosage units comprising effective amounts of lnterleukin-2 and a methylol transfer agent for inhibiting tumor metastasis in a cancer patient, as well as to pharmaceutical compositions comprising such combinations.

The invention is further illustrated by the following non-limiting examples.

### EXAMPLE 1

A 63 year old patient diagnosed with metastatic malignant melanoma was treated as follows.

| | |
|---|---|
| Presentation | Right supra-clavicular mass. Originally had nodular |
| | melanoma excised from right elbow, and had high-dose |
| | interferon post-operatively. Required axillary clearance |
| | for a mass in right axilla eight months later. Further |
| | staging was clear at that time. Presented one year |
| | later with a fixed inoperable mass in right supra- |
| | clavicular area. |
| Treatment | IL-2 and Taurolidine |
| Regimen | Interleukin-2 |
| | Day 1: 18 million units/m² IL-2 infusion over 6 hours |
| | Day 2: 18 million units/m² IL-2 infusion over 12 hours |
| | Day 3: 18 million units/m² IL-2 infusion over 24 hours |
| | Days 4-7: 18 million units/m² IL-2 infusion over 78 hours |
| | Taurolidine |
| | Taurolidine 2% 250 ml infusion over twelve hours, daily |
| | during IL-2 administration |
| | Completed five courses of the above |

After one year, the patient is alive and well, with no evidence of disease on imaging.

### EXAMPLE 2

A 50 year old patient diagnosed with metastatic renal cell carcinoma was treated as follows.

| | |
|---|---|
| Presentation | Haemoptysis - 20 to pulmonary metastases. Noted to |
| | have hepatic metastases, in addition to a iarge mass in |
| | the left kidney. |
| Treatment | IL-2 and Taurolidine |
| Regimen | Interleukin-2 |
| | Day 1: 18 million units/m² IL-2 infusion over 6 hours |
| | Day 2: 18 million units/m² IL-2 infusion over 12 hours |
| | Day 3: 18 million units/m² IL-2 infusion over 24 hours |
| | Days 4-7: 18 million units/m² IL-2 infusion over 78 hours |
| | Tauroldine |
| | Taurolidine 2% 250 ml infusion over two hours, twice |
| | daily during IL-2 administration |
| | Completed five courses of the above |
| Further | Left radical nephrectomy |
| treatment | |

After five years, the patient is alive and well, with no evidence of disease on imaging.

### EXAMPLE 3

A male patient who had recurrent nodular melanoma after interferon treatment was subsequently treated with Interleukin-2 and Taurolidine as follows:

| | |
|---|---|
| Presentation | Recurrence of nodular melanoma lesion in right |
| | shoulder. |
| Treatment | IL-2 and Taurolidine |
| Regimen | Interleukin-2 |
| | Day 1: 36 million units/m² IL-2 infusion over 6 hours |
| | Day 2: 36 million units/m² IL-2 infusion over 12 hours |
| | Day 3: 36 million units/m² IL-2 infusion over 24 hours |
| | Days 4-7: 36 million units/m² IL-2 infusion over 78 hours |
| | Taurolidine |
| | Taurolidine 2% 250 ml infusion over twelve hours, |
| | sequentially with IL-2 administration, during days 1-6 |
| | Undertook five courses - - - during second course, |
| | treatment was interrupted and stopped at 78 hours, |
| | and during the fifth course, treatment was interrupted |
| | during day 4. |

Follow-up CT scans indicated a reduction in the size of the lesion, and subsequently indicated no evidence of disease.

1. A method of inhibiting tumor metastasis in a cancer patient comprising administering to said patient a combination therapy comprising effective amounts of IL-2 and a methylol transfer agent.

2. The method of claim 1 wherein said tumor is a lymphoma, carcinoma or sarcoma.

3. The method of claim 1 wherein said tumor is a glioma, a neuroblastoma, an astrocytoma, carcinomatous meningitis, breast cancer, ovarian cancer, colon cancer, prostate cancer, pancreatic cancer, CNS cancer, liver cancer, lung cancer, gastric cancer, esophageal cancer, urinary bladder cancer, leukemia, melanoma, and renal cell cancer.

4. The method of claim 1 wherein said methylol transfer agent is taurolidine, taurultam, a biologically active derivative thereof, or a mixture thereof.

5. The method of claim 1 wherein said methylol transfer agent is taurolidine, taurultam or a mixture thereof.

6. The method of claim 5 wherein said tumor metastasis is metastatic malignant melanoma or metastatic renal cell carcinoma.

7. A method of treating cancer selected from the group consisting of renal cancer and malignant melanoma, in a patient having said cancer, comprising administering to said patient a combination therapy comprising effective amounts of interleukin-2 and a methylol transfer agent.

8. The method of claim 7 wherein said methylol transfer agent is taurolidine, taurultam, a biologically active derivative thereof, or a mixture thereof.

9. The method of claim 7 wherein said methylol transfer agent is taurolidine, taurultam or a mixture thereof.

10. In combination: Interleukin-2 (IL-2) and a methylol transfer agent, in effective amounts for simultaneous, separate or sequential use for inhibiting tumor metastasis in a cancer patient.

11. The combination of claim 10 wherein said IL-2 and said methylol transfer agent are each present as a pharmaceutical dosage unit.

12. The combination of claim 11 wherein said methylol transfer agent is taurolidine, taurultam, a biologically active derivative thereof, or a mixture thereof.

13. The combination of claim 11 wherein said methylol transfer agent is taurolidine, taurultam or a mixture thereof.

14. A pharmaceutical combination comprising each of the pharmaceutical dosage units of claim 13, for use in inhibiting tumor metastasis in a cancer patient.

15. A pharmaceutical composition for use in inhibiting tumor metastasis in a cancer patient, comprising the combination of claim 14.
